(19) United States
(12) Patent Application Publication
(10) Pub. No.: US 2022/0248647 A1
(43) Pub. Date: Aug. 11, 2022

(54) TRANSGENIC CLONED PIG FOR XENOTRANSPLANTATION EXPRESSING HUMAN CD46 AND TBM GENES, IN WHICH PORCINE ENDOGENOUS RETROVIRUS ENVELOPE C IS NEGATIVE AND GGTA1, CMAH, iGb3s AND ß4GalNT2 GENES ARE KNOCKED OUT, AND METHOD FOR PREPARING SAME

(71) Applicant: Optipharm Co., Ltd., Chungcheongbuk-do (KR)

(72) Inventors: Ki Myung CHOI, Sejong-si (KR); Joo Hyun SHIM, Sejong-si (KR); Na Young KO, Gyeonggi-do (KR); Hyoung Joo KIM, Sejong-si (KR); Yong Jin LEE, Sejong-si (KR); Jae Kyung PARK, Chungcheongbuk-do (KR); Kyung Min Min KWAK, Chungcheongbuk-do (KR); Hyun II KIM, Seoul (KR)

(21) Appl. No.: 17/629,770

(22) PCT Filed: Jul. 23, 2020
(86) PCT No.: PCT/KR2020/009716

§ 371 Date: Jan. 24, 2022

(30) Foreign Application Priority Data
Jul. 23, 2019 (KR) 10-2019-0088960

(51) Int. Cl. A01K 67/027; C07K 14/745; C12N 15/85; C07K 14/705; C12N 9/10; C12N 9/02

(52) U.S. Cl. CPC A01K 67/0276; C07K 14/7455; C12N 15/8509; C07K 14/70596; C12N 9/1051; C12Y 204/01087; A01K 2217/075; A01K 2227/108; A01K 2267/025; C12Y 114/18002; C12N 9/0071

(57) ABSTRACT

The present invention relates to a transgenic cloned pig for xenotransplantation in which porcine endogenous retrovirus (RUN) EnvC is negative, α1,3-galactosyltransferase (GGTA1), CMP-N-acetylneuraminic acid hydroxylase (CMAH), isoglobotrihexosylceramide synthase (iGb3s), and beta-I,4-N-acetyl-galactosaminyl transferase2 (β4GalNT2) are knocked out, and human CD46 and thrombomodulin (TBM) genes are expressed, and to a method of preparing the transgenic cloned pig. The transgenic cloned pig according to the present invention may overcome hyperacute and antigen-antibody mediated immune rejection reaction, immune rejection reaction due to blood coagulation, and immune rejection reaction due to complement activity, without causing transfer of porcine endogenous retrovirus that occurs in xenotransplantation. Therefore, the transgenic cloned pig according to the present invention may be usefully utilized as a donor animal for xenotransplantation of organs and cells.

### TECHNICAL FIELD

The present disclosure relates to a transgenic cloned pig for xenotransplantation expressing human CD46 and TBM (Thrombomodulin) genes, in which PERV (Porcine Endogenous Retrovirus) EnvC is negative, and GGTA1 (α1,3-galactosyltransferase), CMAH (CMP-N-acetylneuraminic acid hydroxylase), iGb3s (Isoglobotrihexosylceramide synthase) and β4GalNT2 (Beta-1,4-N-Acetyl-Galactosaminyl Transferase2) are knocked out, and to a method for preparing the same.

### BACKGROUND ART

As of 2017, there were 27,701 people waiting for organ transplants in Korea, compared to 1,693 donors. Although the nation is actively promoting the need for organ donation and promoting the organ donation culture via courtesy to the bereaved family, a gap between supply and demand continues to increase every year. This is a big problem not only in Korea but also around the world. Thus, illegal organ trading is prevalent.

Xenotransplantation in which organs from other species replace living organs of humans is one of the solutions expected to eradicate the organ supply problem. Among heterogeneous organ source animal models, the organs of mini-pigs are morphologically and genetically similar to those of humans and have been verified in several literatures. In particular, the Yucatan miniature pig is the most used experimental animal model together with the Gottingen miniature pig, and many research results thereon have been derived accordingly. However, according to the research results so far, when organs of mini-pigs are transplanted into humans, there are a number of problems that may cause a much more serious immune rejection reaction than when autologous or allogeneic transplantation is employed.

Among factors that cause immune rejection reaction, α-gal a-galactosyltransferse) is an antigen synthesized by the GGTA1 gene and is present on the cell surface of all animals, including mammals and rodents, except primates. Therefore, when organs from pigs with α-gal are transplanted into humans without α-gal, tissue necrosis and death due to antigen-antibody reaction occur. Therefore, a study on preparation of transgenic cloned pigs deficient in the α-gal was conducted. In 2005, it was reported that when organs from transgenic cloned pigs deficient in GGTA1 gene were transplanted into monkeys in a homozygous manner, the monkeys survived without hyperacute immune rejection reaction. The hyperacute immune rejection reaction that occurs in seconds to minutes was controlled via the preparation of transgenic cloned pigs deficient in the GGTA1 gene. However, a survival period of recipients due to acute and cellular immune rejection reaction was not long. Among the genes that cause the immune rejection reaction, CMAH (Cytidine monophosphate-N-acetylneuraminic acid hydroxylase) is a gene that synthesizes Neu5Ac into Neu5Gc. CMAH is present in all living things, including primates and mammals except humans. However, in the human, CMAH gene was modified such that Neu5Gc is not synthesized. Accordingly, Neu5Gc acts as an antigen in the human body. In the organ transplantation, immune rejection reaction due to antigen-antibody reaction occurs. In addition, iGb3s (Isogloboside 3 synthease; A3GalT2) gene is a glycosyl transferase that adds galactose to lactosyl ceramide to synthesize iGb3 as a glycosphingolipid as a composite lipid. iGb3s is known as an alternative route to produce α-gal antigen synthesized by the GGTA1 gene. The β4GalNT2 (Beta-1,4-N-acetyl-galactosaminyltransferase 2) gene is a gene that produces sugar chains. The β4GalNT2 produces GalNAcβ1-4, Gaβ1-4GlcNAcβ1-3Gal, Sd(a) (Sid blood group; CAD or CT) antigen. It has been reported that the β4GalNT2 gene causes cell lysis by complement activity and immune rejection reaction due to non-gal.

Further, hyperacute and acute immune rejection reaction may be controlled via control of antigen-antibody mediated immune rejection reaction. Further, immune rejection reaction due to blood coagulation and human complement activity occurs when pig organs are transplanted into humans. In this regard, the CD46 (Membrane Cofactor Protein; MCP) gene is a surface membrane glycoprotein. The MCP binds to C3b or C4b of the complement activation component on the surface membrane and acts as a cofactor to promote degradation of C3b or C3b to exhibit an inhibitory effect of the complement activity. In addition, the thrombomodulin (TBM) gene binds to thrombin in the blood coagulation pathway to generate the Thrombomodulin-Thrombin complex and activates protein C, thereby inhibiting blood coagulation due to factor V and factor VII activity.

### DISCLOSURE

### Technical Problem

Under the above background, the present inventors have continued efforts to develop transgenic cloned pigs that may be used for xenotransplantation. We prepared a transgenic cloned pig for xenotransplantation expressing human CD46 and TBM (Thrombomodulin) genes, in which PERV (Porcine Endogenous Retrovirus) EnvC is negative, and GGTA1 (α1,3-galactosyltransferase), CMAH (CMP-N-acetylneuraminic acid hydroxylase), iGb3s (Isoglobotrihexosylceramide synthase) and β4GalNT2 (Beta-1,4-N-Acetyl-Galactosaminyl Transferase2) are knocked out. When using the transgenic cloned pig, this does not cause the problem of metastasis of porcine endogenous retrovirus that occurs in xenotransplantation using the conventionally developed transgenic cloned pig, and overcomes hyperacute and antigen-antibody-mediated immune rejection reaction, immune rejection reaction due to blood coagulation, and immune rejection reaction due to complement activity. We have identified that the transgenic cloned pig has an excellent effect to increase the survival period of recipients. In this way, we completed the present disclosure.

Thus, a purpose of the present disclosure relates to a transgenic cloned pig for xenotransplantation expressing human CD46 and TBM (Thrombomodulin) genes, in which PERV (Porcine Endogenous Retrovirus) EnvC is negative, and GGTA1 (α1,3-galactosyltransferase), CMAH (CMP-N-acetylneuraminic acid hydroxylase), iGb3s (Isoglobotrihexosylceramide synthase) and β4GalNT2 (Beta-1,4-N-Acetyl-Galactosaminyl Transferase2) are knocked out, and to provide a method for preparing the same.

### Technical Solution

In order to achieve the above purpose, the present disclosure provides a transformed cell for preparing a transgenic cloned pig for xenotransplantation into which a recombinant vector for knocking out GGTA1 (Alpha 1,3-Galactosyltransferase), a recombinant vector for knocking out CMAH (CMP-N-acetylneuraminic acid hydroxylase), a recombinant vector for knocking out iGb3s (Isoglobotrihexosylceramide synthase), a recombinant vector for knocking out β4GalNT2 (Beta-1,4-N-Acetyl-Galactosaminyl Transferase2), a recombinant vector for expressing human CD46 and a recombinant vector for expressing human TBM (Thrombomodulin) are introduced and in which PERV (Porcine Endogenous Retrovirus) EnvC (Envelope C) is negative.

Further, the present disclosure provides a method for preparing a transgenic cloned pig for xenotransplantation, the method including a step of transplanting the transformed cell into an enucleated oocyte to prepare a nuclear transferred oocyte; and a step of transplanting the nuclear transferred oocyte into a fallopian tube of a surrogate mother.

Further, the present disclosure provides a transgenic cloned pig for xenotransplantation as prepared by the above method.

### Advantageous Effects

In the transgenic cloned pig according to the present disclosure, the porcine endogenous retrovirus EnvC is negative, and the four genes, that is, GGTA1, CMAH, β4GalNT2 and iGb3s are knocked out by CRISPR-Cas9, a gene scissors. The transgenic cloned pig expresses human CD46 and TBM genes. Accordingly, the transgenic cloned pig according to the present disclosure may overcome hyperacute and antigen-antibody mediated immune rejection reaction, immune rejection reaction due to blood coagulation, and immune rejection reaction due to complement activity, without causing transfer of porcine endogenous retrovirus that occurs in xenotransplantation. Therefore, the transgenic cloned pig according to the present disclosure may be usefully utilized as a donor animal for xenotransplantation of organs and cells.

### DESCRIPTION OF DRAWINGS

FIG. 1 is a diagram showing a targeting vector for deletion of GGTA1, CMAH, iGb3s, β4GalNT2 genes.

FIG. 2 is a diagram showing a vector map for human CD46 expression.

FIG. 3 is a diagram showing a vector map for human TBM expression.

FIG. 4 is a diagram showing the results of the porcine endogenous retrovirus envelope C test.

FIG. 5 is a diagram showing the results of immunofluorescence staining and cell sorting using human CD46 antibody after transduction.

FIG. 6 is a diagram identifying the presence or absence of introduction of human CD46 and TBM expression vectors into transformed cells.

FIG. 7 is a diagram showing the nucleotide sequences of GGTA1, CMAH, iGb3s, and β4GalNT2 genes in the transformed cell.

FIG. 8 is a diagram showing the results of identifying whether colony porcine endogenous retrovirus envelope C is negative in a transformed cell line #18 via DNA and RNA analysis.

FIG. 9 is a diagram showing the observation result based on a fluorescence microscope of colony immunofluorescence staining of the transformed cell line #18.

FIG. 10 is a diagram showing the results of identification via FACS analysis of colony immunofluorescence staining of the transformed cell line #18.

FIG. 11 is a diagram showing the Western blot results of the transformed cell line #18 colony.

FIG. 12 is a photograph of transgenic cloned pigs prepared via somatic cell nuclear transfer using the transformed cell line #18.

FIG. 13 is a diagram showing gene analysis of transgenic cloned pigs prepared via somatic cell nuclear transfer using the transformed cell line #18.

FIG. 14 is a diagram showing the results of FACS analysis of immunofluorescence staining using PBMCs derived from blood of transgenic cloned pig as prepared via somatic cell nuclear transfer using the transformed cell line #18.

FIG. 15 is a diagram showing the Western blot results using ear fibroblasts of transgenic cloned pigs as prepared via somatic cell nuclear transfer using the transformed cell line #18.

FIG. 16 is a diagram showing the results of FACS analysis of immunofluorescence staining using corneal endothelial cells of transgenic cloned pigs as prepared via somatic cell nuclear transfer using the transformed cell line #18.

FIG. 17 is a diagram showing the results of tissue immunofluorescence staining using organs of transgenic cloned pigs as prepared via somatic cell nuclear transfer using the transformed cell line #18.

FIG. 18 is a diagram showing the results of APC (Activated Protein C) quantification in spleen cells of transgenic cloned pigs as prepared via somatic cell nuclear transfer using the transformed cell line #18.

FIG. 19 is a diagram showing the results of C3 deposition analysis using ear fibroblasts of transgenic cloned pigs as prepared via somatic cell nuclear transfer using the transformed cell line #18.

### MODES OF THE INVENTION

Hereinafter, the present disclosure will be described in more detail.

In one aspect, the present disclosure provides a transformed cell for preparing a transgenic cloned pig for xenotransplantation into which a recombinant vector for knocking out GGTA1 (Alpha 1,3-Galactosyltransferase), a recombinant vector for knocking out CMAH (CMP-N-acetylneuraminic acid hydroxylase), a recombinant vector for knocking out iGb3s (Isoglobotrihexosylceramide synthase), a recombinant vector for knocking out β4GalNT2 (Beta-1,4-N-Acetyl-Galactosaminyl Transferase2), a recombinant vector for expressing human CD46 and a recombinant vector for expressing human TBM (Thrombomodulin) and in which PERV (Porcine Endogenous Retrovirus) EnvC (Envelope C) is negative.

In the present disclosure, a “vector” refers to a gene construct including the nucleotide sequence of a gene operably linked to a suitable regulatory sequence so as to express a target gene in a suitable host. The regulatory sequence may include a promoter capable of initiating transcription, any operator sequence for regulating such transcription, and a sequence regulating the termination of transcription and translation. The vector according to the present disclosure is not particularly limited as long as it is capable of replication in a cell. Any vector known in the art may be used, for example, a plasmid, cosmid, phage particle, or viral vector.

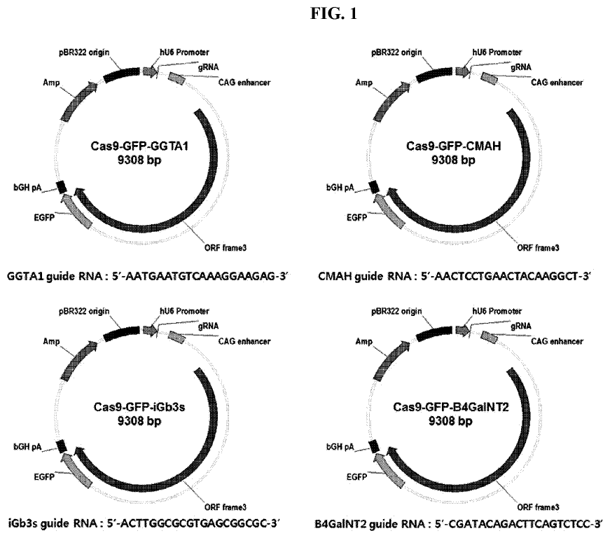

In the present disclosure, the recombinant vectors for knocking out may be configured such that all of the nucleotide sequences encoding the sgRNAs relative to GGTA1 (Alpha 1,3-Galactosyltransferase), CMAH (CMP-N-acetylneuraminic acid hydroxylase), iGb3s (Isoglobotrihexosylceramide synthase) and β4GalNT2 (Beta-1,4-N-Acetyl-Galactosaminyl Transferase2) are included in one vector, or such that at least one nucleotide sequence encoding each of the sgRNAs relative to each of GGTA1 (Alpha 1,3-Galactosyltransferase), CMAH (CMP-N-acetylneuraminic acid hydroxylase), iGb3s (Isoglobotrihexosylceramide synthase) and β4GalNT2 (Beta-1,4-N-Acetyl-Galactosaminyl Transferase2) is included in a separate vector. Hereinafter, as long as the vector includes a target sequence, the disclosure is not limited to a configuration and the number of vectors. In one example of the present disclosure, the four recombinant vectors for knocking out respectively including the sgRNAs relative to GGTA1 (Alpha 1,3-Galactosyltransferase), CMAH (CMP-N-acetylneuraminic acid hydroxylase), iGb3s (Isoglobotrihexosylceramide synthase) and β4GalNT2 (Beta-1,4-N-Acetyl-Galactosaminyl Transferase2) may be used. A specific vector map thereof is shown in FIG. 1.

In the present disclosure, the ‘GGTA1 (alpha 1,3-galactosyltransferase)’ gene is responsible for the biosynthesis of α-Gal. The pig has 8 introns and 9 exons. The GGTA1 gene may be GenBank accession No. AH010595.2.

The recombinant vector for knocking out the GGTA1 is characterized by recognizing the nucleotide sequence site represented by SEQ ID NO: 1 located at exon #4 of porcine chromosome 1, that is, a guide sequence site.

In the present disclosure, the ‘CMAH (CMP-N-acetylneuraminic acid hydroxlase)’ gene is responsible for the biosynthesis of Neu5Gc. The CMAH gene may be GenBank accession No. NM_001113015.1.

The recombinant vector for knocking out the CMAH is characterized by recognizing the nucleotide sequence site represented by SEQ ID NO: 2 located at exon #9 of porcine chromosome 7, that is, the guide sequence site.

In the present disclosure, the ‘iGb3s (Isogloboside 3 synthase)’ gene synthesizes igb3, a glycosphingolipid. The iGb3s gene may be Genbank accession No. XM_021095855.

The recombinant vector for knocking out the iGb3s is characterized by recognizing the nucleotide sequence site represented by SEQ ID NO: 3 located at exon #4 of porcine chromosome 6, that is, the guide sequence site.

In the present disclosure, the ‘β4GalNT2(beta-1,4-N-acetyl-galactosaminyltransferase 2)’ gene synthesizes an SD^{a} antigen. The β4GalNT2 gene may be Genbank accession No. NM_001244330.1.

The recombinant vector for knocking out the β4GalNT2 is characterized by recognizing the nucleotide sequence site represented by SEQ ID NO: 4 located in exon #1 of porcine chromosome 12, that is, the guide sequence site.

In the present disclosure, the gRNA may be an RNA that may produce a composite with a Cas9 protein, and bring a Cas protein to the target DNA. For example, the gRNA may be transcribed from the DNA represented by SEQ ID NOs: 1 to 4. In other words, in the present disclosure, a gRNA sequence and a DNA sequence corresponding thereto are used interchangeably with each other. It is obvious to those skilled in the art that the gRNA is included in a vector and expressed via transcription, and thus may be experimentally described as a DNA sequence.

In the present disclosure, ‘CRISPR-Cas9’ is a type of gene scissors and is used for cloning for gene removal. In the present disclosure, the Cas9 protein refers to an essential protein element in the CRISPR/Cas9 system. When producing a composite with two RNAs called CRISPR RNA (crRNA) and transactivating crRNA (tracrRNA), the Cas9 protein produces an active endonuclease or nickase. Genes encoding Cas9 proteins are generally associated with CRISPR repeat-spacer arrays, and there are 40 or more different Cas protein families. Representatively, there are three types of CRISPR-Cas systems. Among them, a type II CRISPR/Cas system involving Cas9 protein is representative.

In the present disclosure, ‘gene scissors’ refers to means that cuts DNA at a desired site in the genome, and refers to a genome editing scheme that recognizes a specific nucleotide sequence in the genome and precisely cuts the DNA of the recognized specific nucleotide sequence.

Recombinant vectors for knocking out according to the present disclosure include Streptococcus pyogenes-derived SpCas9 for DNA cleavage in addition to gRNA (guide RNA) related to DNA binding. The gRNA domain may include a cloning site that may bind to any sequence in DNA and thus may bind to a desired specific sequence of genomic DNA. DNA cleavage is induced via guide of gRNA bound to a specific site and activity of Cas9 protein.

In the present disclosure, hCD46 (Membrane Cofactor Protein; MCP) gene is responsible for inhibiting complement activity.

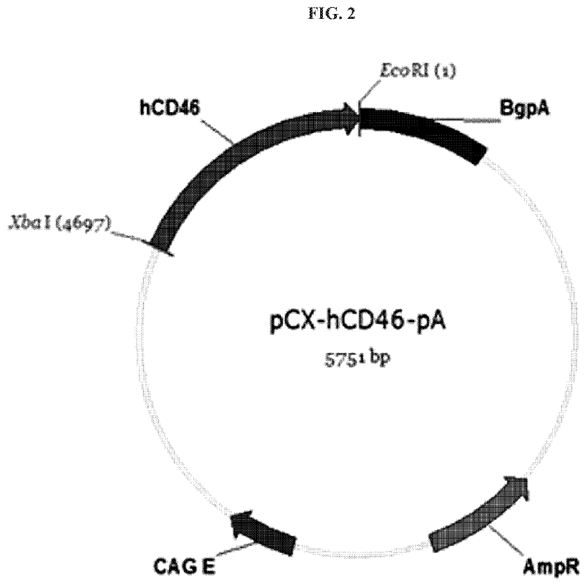

The recombinant vector for expressing human CD46 is intended for introducing the human CD46 gene. For example, the recombinant vector for expressing human CD46 may be a vector composed of the vector map shown in FIG. 2. However, the disclosure is not limited thereto.

In the present disclosure, hTBM (Thrombomodulin) gene is responsible for blood coagulation inhibition.

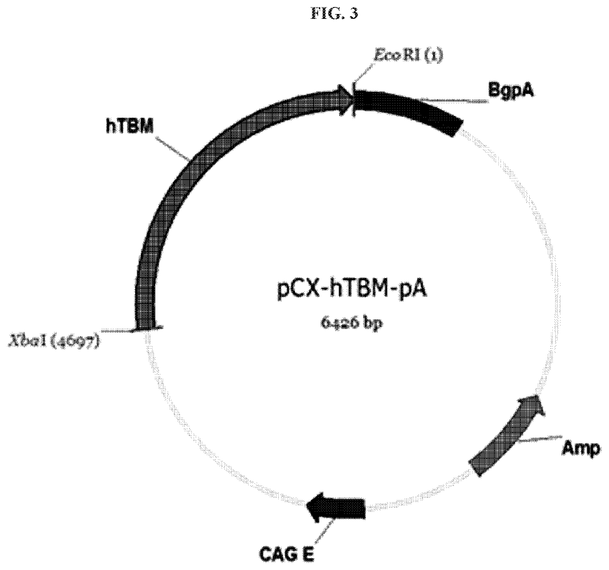

The recombinant vector for expressing the human TBM (Thrombomodulin) is intended for introducing the human TBM gene. For example, the recombinant vector for expressing the human TBM (Thrombomodulin) may be a vector composed of the vector map shown in FIG. 3. However, the disclosure is not limited thereto.

The vector according to the present disclosure may include a primer sequence, for example, a CAG promoter. In addition to this promoter, promoters that may be expressed in mammals, such as the EF1α promoter, which are generally considered equivalent to the CAG promoter may be used. Further, mammalian tissue-specific promoters such as the ICAM2 promoter may also be used. The CAG is one of gene expression promoters and is used for foreign gene expression.

In the present disclosure, the ‘promoter’ may generally act as the transcription initiation point and may be located in front of the DNA nucleotide sequence carrying the genetic information of the gene to be expressed. The promoter is located within several hundred bases from a transcription start point. In eukaryotes, a protein called a transcriptional regulator binds to a promoter region and thus is involved in the binding of RNA polymerase.

In the present disclosure, “transformation” means introducing DNA into a host so that the DNA becomes replicable as an extrachromosomal factor or by chromosomal integrity completion. Transformation includes any method of introducing a nucleic acid molecule into an organism, cell, tissue or organ. As is known in the art, the transformation may be performed by selecting an appropriate standard technique according to the host cell. For example, the transformation may include electroporation, calcium phosphate (CaPO₄) precipitation, calcium chloride (CaCl₂) precipitation, microinjection, polyethylene glycol (PEG) method, DEAE-dextran method, cationic liposome method, and lithium acetate-DMSO method. However, the disclosure is not limited thereto. In order to distinguish the transformation of eukaryotic cells by plasmid or non-plasmid naked DNA from transformation in the sense of tumorigenesis of cells, the transformation may be referred to as ‘transfection’. However, in the present disclosure, both have the same meaning.

The transformed cell is preferably a fibroblast, more preferably a porcine fibroblast, but is not limited thereto.

The transformed cell according to the present disclosure may be a cell with accession number KCLRF-BP-00464 deposited with the Korea Cell Line Research Foundation (KCLRF) on Jan. 30, 2019.

In another aspect, the present disclosure provides a method for preparing a transgenic cloned pig for xenotransplantation, the method including a step of transplanting the transformed cell into an enucleated oocyte to prepare a nuclear transferred oocyte; and a step of transplanting the nuclear transferred oocyte into a fallopian tube of a surrogate mother, and a transgenic cloned pig for xenotransplantation prepared by the above method.

In the present disclosure, ‘nuclear transfer’ refers to a genetic manipulation technique that artificially combines nuclear DNA of another cell to a cell without a nucleus to have the same trait. The nuclear transfer may employ a method known in the art.

In the present disclosure, ‘nuclear transferred oocyte’ refers to an oocyte into which a donor nuclear source cell is introduced or fused.

In the present disclosure, ‘enucleated oocyte’ means that the nucleus of the oocyte has been removed.

In the transgenic cloned pig according to the present disclosure, porcine endogenous retrovirus EnvC is negative, two loci of GGTA1, CMAH, and β4GalNT2 genes and one locus of iGb3s gene are removed by CRISPR-Cas9 system as a gene scissors. The transgenic cloned pig has the characteristics of expressing the human CD46 and TBM genes. Thus, the transgenic cloned pig according to the present disclosure may overcome hyperacute and antigen-antibody-mediated immune rejection reaction, immune rejection reaction due to blood coagulation, and immune rejection reaction due to complement activity while not causing metastasis of porcine endogenous retrovirus that occurs in xenotransplantation.

Therefore, the transgenic cloned pig according to the present disclosure may be usefully utilized as a donor animal for transplantation of heterogeneous organs and cells.

Hereinafter, the present disclosure will be described in detail by way of example. The following examples are only for illustrating the present disclosure, and the present disclosure is not limited by the following examples.

### EXAMPLES

### Example 1. Preparation of GGTA1, CMAH, iGb3s and β4GalNT2 Gene Targeting Vectors

To knock out the porcine GGTA1, CMAH, iGb3s and β4GalNT2 genes, the nucleotide sequence of each of the genes was analyzed. Then, a nucleotide sequence site of the exon to which gRNA may bind was determined based on the analysis result. In this connection, gRNAs for gene targeting did not simply use known gRNAs. Rather, via a screening process, the exon site that may maximize gene targeting efficiency was determined, and gRNA having excellent effects at the corresponding exon site was selected. The gRNA selected via the above process was synthesized by Bioneer for insertion into the vector (SEQ ID NOs: 1 to 4). Table 1 shows the sequence of the gRNA relative to each of the genes, NCBI accession number, chromosomal location, and exon location.

TABLE 1

| SEQ | | | | | |
|---|---|---|---|---|---|
| [symbol] | Gene | [symbol] | Chromosome | Exon | [symbol] RNA sequence (5′-3′) |
| 1 | GGTA1 | [symbol] | 1 | 4 | AATGAATGTCAAAGGAAGAG |
| | [symbol] | | | | |
| 2 | CMAH | NM_0011130015.1 | 7 | 9 | AACTCCTGAACTACAAGGCT |
| | [symbol] | | | | |
| | [symbol] | | | | |
| 3 | [symbol] | [symbol] | 6 | 4 | ACTTGGCGCGTGAGCGGCGC |
| | [symbol] | | | | |
| 4 | β4GalNT2 | [symbol] | 22 | 1 | CGATACAGACTTCAGTCTCC |
| | [symbol] | | | | |
| | 2) | | | | |

[symbol]

indicates data missing or illegible when filed

Two primers for each gene shown in Table 2 were hybridized with each other so as to include the gRNA nucleotide sequence capable of binding to the porcine GGTA1, CMAH, iGb3s and β4GalNT2 exon nucleotide sequence sites disclosed in Table 1. Then, the product was inserted into a Cas9-GFP vector. More specifically, 100 pmol of the two primers for each gene were mixed with each other, and the temperature was lowered for 10 minutes at 95° C. and for 10 minutes at 85° C. by 0.1° C. per second to 12° C. to perform hybridization. A Cas9-GFP vector obtained by cutting the hybridized product with the restriction enzyme Bbsl was used as a template. Ligation and transformation were performed on each gene using T4 DNA ligase (NEB). Sequence analysis of the completed vector was performed to identify absence or presence of the introduction of the guide RNA sequence. The vector map is shown in FIG. 1 (Genotech).

TABLE 2

| SEQ | | |
|---|---|---|
| IN NO. | Target gene | Primer Sequence (5′-3′) |
| 5 | GGTA1 | CACCAATGAATGTCAAAGGAAGAG |
| 6 | | AAACCTCTTCCTTTGACATTCATT |
| 7 | CMAH | CACCAACTCCTGAACTACAAGGCT |
| 8 | | AAACAGCCTTGTAGTTCAGGAGTT |
| 9 | iGb3s | CACCACTTGGCGCGTGAGCGGCGC |
| 10 | | AAACGCGCCGCTCACGCGCCAAGT |
| 11 | β4GalNT2 | CACCCGATACAGACTTCAGTCTCC |
| 12 | | AAACGGAGACTGAAGTCTGTATCG |

### Example 2. Preparing of Human CD46 and TBM Gene Expression Vectors

For the construction of human CD46 and TBM gene expression plasmid vectors, nucleotide sequences of Genbank number D84105.1 (human CD46) and Genbank number J02973.1 (human TBM) were respectively amplified based on the sequences disclosed in NCBI.

More specifically, while a primer (human CD46: forward primer (SEQ ID NO: 13): 5′-TATCTAGAATGGAGCCTCCCGGC-3′, reverse primer (SEQ ID NO: 14): 5′-CGGATATCTATTCAGCCTCTCTGCTCTGCTGGA-3; Human TBM: forward primer (SEQ ID NO: 15): 5′-CCTGGGTAACGATATCATGCTTGGGG-3′, reverse primer (SEQ ID NO: 16): 5′-GACGGAGGCCGAATTCGCTCAGAGTC-3′) with an XbaI restriction enzyme sequence inserted into the 5′ terminal thereof and an EcoRI restriction enzyme sequence inserted into the 3′ terminal thereof for cloning using a restriction enzyme, and human cDNA library were used as a template, gene amplification was executed using pfu taq polymerase. After A-tailing each PCR product as prepared, TA-cloning was performed thereon. After nucleotide sequence analysis, the plasmid DNA cloned without deformation was inserted into the pCX vector cut with XbaI and EcoRI. A schematic diagram of the constructed recombinant vector is shown in FIG. 2 and FIG. 3, respectively.

### Example 3. Selection of Individual in which Porcine Endogenous Retrovirus Envelope C is Negative

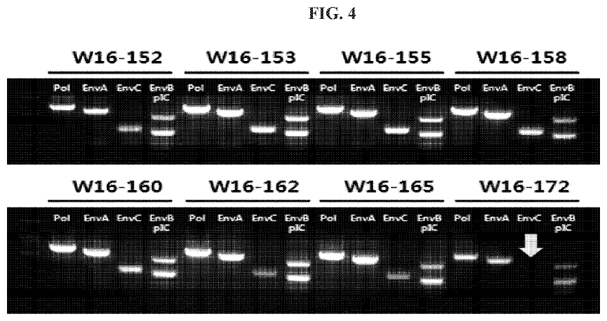

In order to select an individual in which the porcine endogenous retrovirus envelope C was negative, genomic DNA was extracted from each individual, and then PCR was performed using the primer pairs shown in Table 3. More specifically, after obtaining ear tissues for each individual, each genomic DNA was extracted therefrom using the Dneasy Blood & tissue kit (QIAGEN, Germany). After reacting using the extracted genomic DNA and primers for initial denaturation at 95° C. for 5 minutes, a total of 35 cycles of 40 seconds at 95° C., 40 seconds at 61° C., and 1 minute at 72° C. were repeated. Finally, the reaction was carried out at 72° C. for 7 minutes. The PCR results were loaded on a 2% agarose TAE gel, and the results are shown in FIG. 4.

TABLE 3

| Gene | Primer (5′-3′) | Size (bp) | SEQ ID NO. |
|---|---|---|---|
| PERV [symbol] | F: GGAAGCAGCTATGTGGTGCAAG | 708 | 17 |
| | R: CACAATGTTTGACCACCCAGTC | | 18 |
| PERV EnvA | F: CTGCCTTCGATCAGTAATCCCT | 606 | 19 |
| | R: GGGGACTGATCCAGAGGTTGTA | | 20 |
| PERV EnvB | F: CTGTGGGGGTTCTGGGGAA | 454 | 21 |
| | R: GGTACCGTTGCTAGGCGGCT | | 22 |
| PERV EnvC | F: TCTATACGTTTGCCTCAGATCAGT | 251 | 23 |
| | R: CCAGGTCAGGTAATTAAATTGTCC | | 24 |
| [symbol] internal | F: CTGAGGAGCTACGGTCATCACAA | 200 | 25 |
| control | R: TAGGGTTGTTGGATCCGGTTTC | | 26 |

[symbol]

indicates data missing or illegible when filed

As shown in FIG. 4, it was identified that the porcine endogenous virus envelope C was negative in W16-172 individuals. Ear fibroblasts were isolated from the W16-172 individual, and then used as template cells for the preparation of transgenic cloned pigs.

### Example 4. Construction of Transformed Cell Line in which GGTA1, CMAH, iGb3s and β4GalNT2 Genes are Removed and which Expresses Human CD46 and TBM Genes

4-1. Preparing of Transformed Cell Line in which GGTA1, CMAH, iGb3s and β4GalNT2 Genes are Removed and which Expresses the Human CD46 and TBM Genes

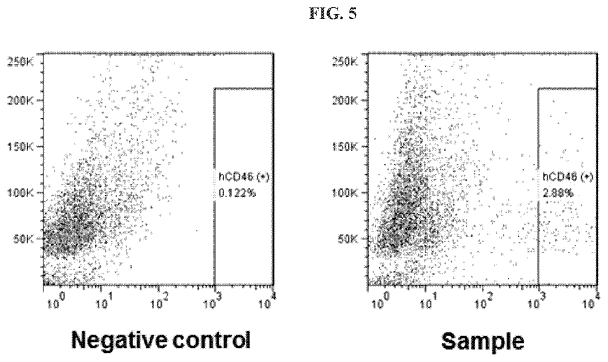

GGTA1, CMAH, iGb3s and β4GalNT2 targeting recombinant vectors prepared in Example 1 were introduced into W16-172 individual-derived fibroblasts in which porcine endogenous retrovirus envelope C was negative as isolated in Example 3, using Lipofectamine 3000 (Invitrogen). After the introduction of the targeting recombinant vectors, only GFP gene-positive cells inserted into the Cas9 vector were first selected using the FACS AriaII equipment. Both of the human CD46 and TBM expression recombinant vectors prepared in Example 2 were introduced into the firstly selected cells using Lipofectamine 3000. To increase selection efficiency, cells were immune-stained using human CD46 antibody after the introduction of the expression vector. The transformed cells were secondarily selected by isolating only human CD46-positive cells using FACS AriaII equipment. This process is shown in FIG. 5.

As shown in FIG. 5, it was identified that only human CD46-positive cells were separated well as a result of FACS AriaII separation.

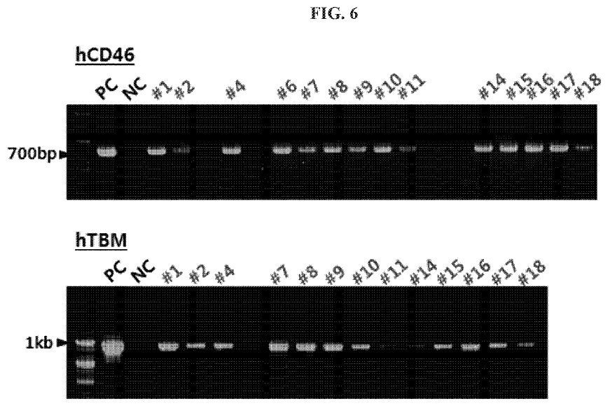

Next, single cell colony culture of the isolated cells was performed using FACS AriaII equipment, and then colony gene analysis was performed. More specifically, after extracting genomic DNA from each transformed cell colony using the Dneasy Blood & tissue kit, PCR was performed using a primer (human CD46 forward primer (SEQ ID NO: 27): CGAGTTTGGTTATCAGATGCA, reverse primer (SEQ ID NO: 28): CGTGCTCTCTCCAATAAGTGA; human TBM forward primer (SEQ ID NO: 29): TACGGGAGACAACAACACCA, reverse primer (SEQ ID NO: 30): AACCGTCGTCCAGGATGTAG) having each position thereof located in the human CD46 and TBM expression recombinant vectors. The obtained PCR product was loaded on a 1% agarose TAE gel, and the results are shown in FIG. 6.

As shown in FIG. 6, it was identified that human CD46 and TBM expression vectors were well inserted in a number of colonies.

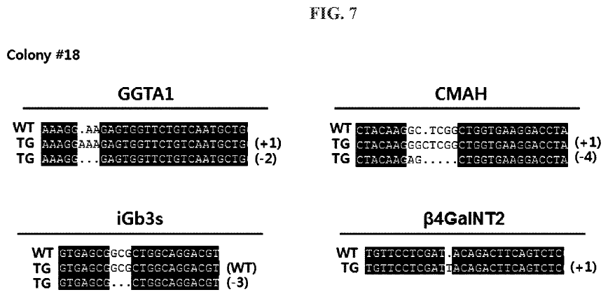

Additionally, PCR was performed using the primer pairs shown in Table 4 so as to include GGTA1, CMAH, iGb3s and β4GalNT2 targeting sites using DNA extracted from the transformed cell colony. The obtained PCR product was provided to Solgent Co., Ltd. which analyzed the nucleotide sequence thereof. The result is shown in FIG. 7.

TABLE 4

| | | Size | SEQ |
|---|---|---|---|
| Gene | Primer (5′-3′) | (bp) | IN NO. |
| GGTA1 | F: CACTTGGTAATTTGCCAGT | 375 | 31 |
| | R: GGTGTCAGTGAATCCTACTT | | 32 |
| CMAH | F: TGTTCTACTTCTGCATCACTC | 378 | 33 |
| | R: CAGCTAAATCACTCATTCAGC | | 34 |
| iGb3s | F: GACAGCAGAGCAGCACTTCAT | 344 | 35 |
| | R: TGTCACGCTCAAAGGGCAGCA | | 36 |
| β4GalNT2 | F: CGTTTGCTCTCTTGTGTC | 250 | 37 |
| | R: AAGTGTCAGTGCAAAGTG | | 38 |

As shown in FIG. 7, it was identified that in the transformed cell line #18 expressing human CD46 and TBM, both loci of GGTA1, CMAH, and β4GalNT2 genes as the target gene sites were deleted, and one locus of the iGb3s gene was deleted.

4-2. Analysis of Porcine Endogenous Retrovirus Envelope C in Selected Transformed Cell Lines

Analysis of porcine endogenous retrovirus envelope C from the transformed cell line #18 selected in Example 4-1 was performed. More specifically, genomic DNA was extracted from the transformed cell line #18 using the Dneasy Blood & tissue kit, and then PCR was performed using the primers listed in the three references. The amplified product was loaded on a 1% agarose TAE gel.

Separately, total RNA was isolated from the transformed cell line #18 selected in Example 4-1 using the Trizol (Ambion) method. Then, cDNA synthesis was performed using the mRNA as a template via RT-PCR premix (Genetbio). Real-time PCR was performed using the synthesized cDNA and the extracted DNA as a template.

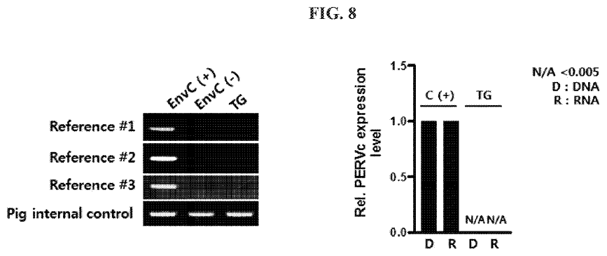

Information on the primer sequences used in the above experiments is shown in Table 5, and the PCR and real-time PCR results are shown in FIG. 8.

TABLE 5

| | Sequence (5′-3′) | Reference | SEQ ID NO. |
|---|---|---|---|
| Reference #1 | TCTATACGTTTGCCTCAGATCAGT | Hyoung-Joon Moon et al., Journal of | 39 |
| | CCAGGTCAGGTAATTAAATTGTCC | veterinary Science | 40 |
| Reference #2 | CACCTATACCAGCTCTGGAC | Seong Lan Yu et al., Journal of | 41 |
| | GTTAGAGGATGGTCCTGGTC | biomedicine and biotechnology, 2012 | 42 |
| Reference #3 | CCCCAACCCAAGGACCAG | Eris Bittmann et al. Virology. 2012 | 43 |
| | AAGTTTTGCCCCCATTTTAGT | | 44 |
| qPERV | CACCTATACCAGCTCTGGACA | [symbol] B et al., Xenotransplantation. | 45 |
| | ATGTTAGAGGATGGTCCTGG | 2009 | 46 |
| GAPDH | ACATGGCCTCCAAGGAGTAAGA | | 47 |
| | GATCGAGTTGGGGCTGTGACT | | 48 |

[symbol]

indicates data missing or illegible when filed

As shown in FIG. 8, it was identified that in the transformed cell line #18, the envelope C was negative at both DNA and RNA levels.

4-3. Analysis of Protein Expression in Selected Transformed Cell Lines

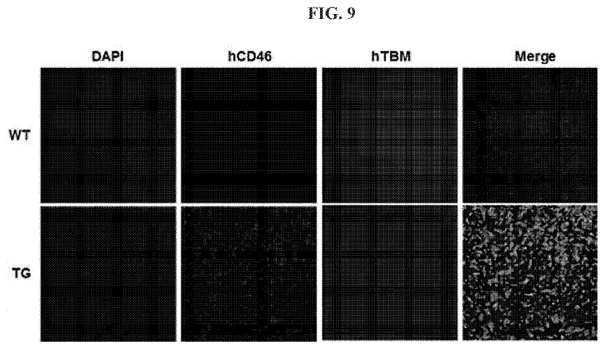

Immunofluorescence staining was performed for protein expression analysis from the #18 colony selected in Example 4-1. More specifically, wildtype (WT) and #18 colony cells were cultured in a 4-well dish containing round glass at 1×10⁴. After washing with DPBS, each of human CD46 antibody, FITC fluorescence-conjugated anti-mouse antibody, and PE fluorescence-conjugated human TBM antibody reacted at a concentration of 1:100 at room temperature for 1 hour. The stained cells were fixed with 1% formalin, and only round glass was separated and analysis thereon was performed using a fluorescence microscope. The results are shown in FIG. 9.

As shown in FIG. 9, it was identified that human CD46 and human TBM proteins were well expressed in the transformed cell line #18 (TG), compared to the wild type.

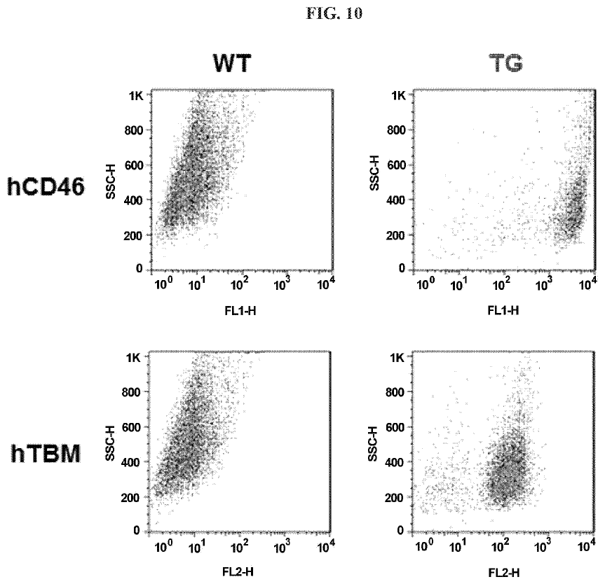

FACS analysis was performed for further analysis of protein expression at the cell level from the #18 colony analyzed via immunofluorescence staining as described above. More specifically, wild-type (WT) and #18 colony cells (TG) were washed with DPBS and treated with 0.25% trypsin-EDTA solution for 3 minutes to obtain cells. Trypsin-EDTA was inactivated using fetal bovine serum (FBS), and the cells were washed with DPBS, and staining of the cells was executed with human CD46 antibody and human TBM antibody. The stained cells were fixed with 1% formalin and were analyzed using FACS caliber II. The results are shown in FIG. 10.

As shown in FIG. 10, it was identified that the human CD46 and TBM proteins were well expressed in the transformed cell line #18 (TG), compared to the wild type.

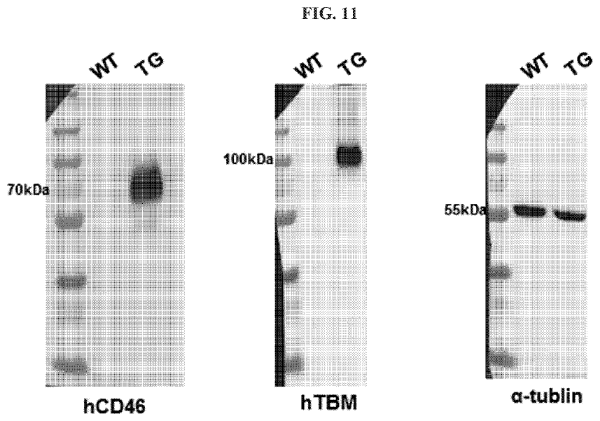

Western-blot analysis was performed to identify additional protein expression. More specifically, wild-type (WT) and #18 colony cells (TG) were treated with RIPA buffer containing proteinase inactivation agent, and crushed using an ultrasonic crusher. After obtaining the supernatant via centrifugation, the supernatant was loaded on an SDS-PAGE gel, which in turn was transferred to a PVDF membrane, which in turn was blocked using 5% skim milk. The blocked membrane was treated with 5% skim milk containing human CD46 antibody and human TBM antibody, and the membrane was treated with a secondary antibody and then reacted. After completion of the reaction, the ECL solution was applied thereto. Analysis was performed using the chemiDoc imaging system (BioRAD). The results are shown in FIG. 11.

As shown in FIG. 11, it was identified that the human CD46 and TBM proteins were expressed in the transformed cell line #18 (TG), compared to the wild type.

The transformed cell line #18 identified via the above experiment was deposited with the Korea Cell Line Research Foundation (KCLRF) on Jan. 30, 2019 under the name of H-01, and was given an accession number KCLRF-BP-00464.

### Example 5. Preparation of Transgenic Pigs in which GGTA1, CMAH, iGb3s and β4GalNT2 Genes are Removed and which Express Human CD46 and TBM Genes

5-1. Preparation of Oocytes

After obtaining the ovaries of immature sows, they were placed in a 0.9% NaCl solution at 35° C. and transported to the laboratory. Cumulus-oocyte complexes (COCs) were aspirated from 2 to 6 mm diameter antral follicles using an 18-gauge needle fixed in a 10 mL disposable syringe. COCs were washed three times with TCM 199 (31100-035, Gibco Grand Island, N.Y., USA) containing 0.1% polyvinyl alcohol, 3.05 mM D-glucose, 0.91 mM sodium pyruvate, 0.57 mM cysteine, 0.5 μg/mL LH (L-5269, Sigma-Aldrich Corp., St. Louis, Mo., USA), 0.5 μg/mL FSH (F-2293, Sigma-Aldrich Corp.), 10 ng/mL epidermal growth factor (E-4127, Sigma-Aldrich Corp.), 75 μg/mL penicillin G, and 50 μg/mL streptomycin. About 50 to 60 COCs were transferred to a 4-well multi-dish (Nunc, Roskilde, Denmark) covered with mineral oil, and 500 mL of the same medium was added thereto. The COCs were cultured at 5% CO₂ and 39° C. conditions.

5-2. Nuclear Transfer

Nuclear transfer was performed with slight modifications to the method of Park et al. (Biol. Reprod. 66:1001-1005, 2002). More specifically, after 42 to 44 hours of culture, oocytes were isolated from cumulus cells by vigorously vortexing the cumulus cells in TL-HEPES containing 0.1% PVA and 0.2% hyaluronidase for 4 minutes. Cell nuclei were removed from cumulus cell-free oocytes by aspirating the first polar body and proximal cytoplasm using a fine glass pipette in TCM 199 containing 0.3% BSA (Sigma-Aldrich Corp., A-8022) and 7.5 μg/mL cytochalasin B. Prior to SCNT, for serum starvation, the donor cells prepared in Example 4 were cultured in DMEM medium containing 0.5% FBS for 3 days. A single donor cell was placed in the perivitelline space of the oocyte in contact with the oocyte membrane. The inoculated oocytes were placed between two 0.2 mm diameter platinum electrodes 1 mm apart from each other in a medium composed of 0.3 M mannitol, 1.0 mM CaCl₂H₂O, 0.1 mM MgCl₂₆H₂O and 0.5 mM HEPES. Fusion/activation was induced by continuously applying a DC pulse of 1.1 kV/cm twice thereto for 30 μs (BTX, USA). Then, 20 to 30 reconstructed embryos were transferred to a 4-well multi-dish covered with mineral oil, and NCSU (North Carolina State University)-23 medium supplemented with 500 mL of 0.4% BSA was added thereto. After 1 or 2 days of culture, NT embryos were surgically implanted into the fallopian tubes of sows, the first day of standing estrus. Pregnancy status was identified with an ultrasound scanner (Mysono 201, Medison Co., LTD, Seoul, Korea).

### Example 6. Verification of Transgenic Pigs in which GGTA1, CMAH, iGb3s and β4GalNT2 Genes are Removed and which Express Human CD46 and TBM Genes

6-1. Identification of Transgenic Cloned Pigs

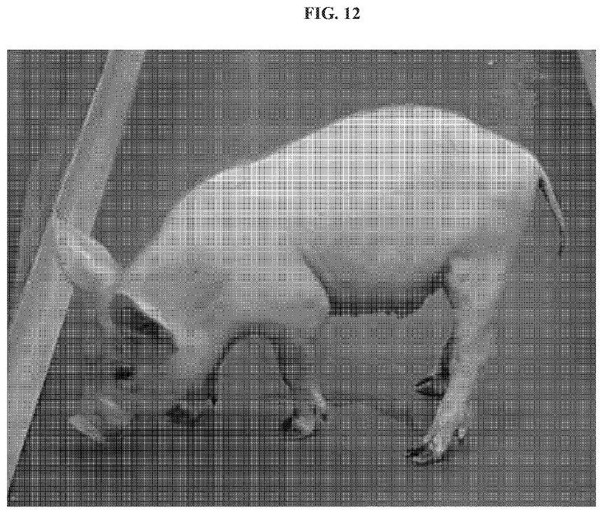

FIG. 12 shows the appearance of the transformed porcine (#1) prepared in Example 5.

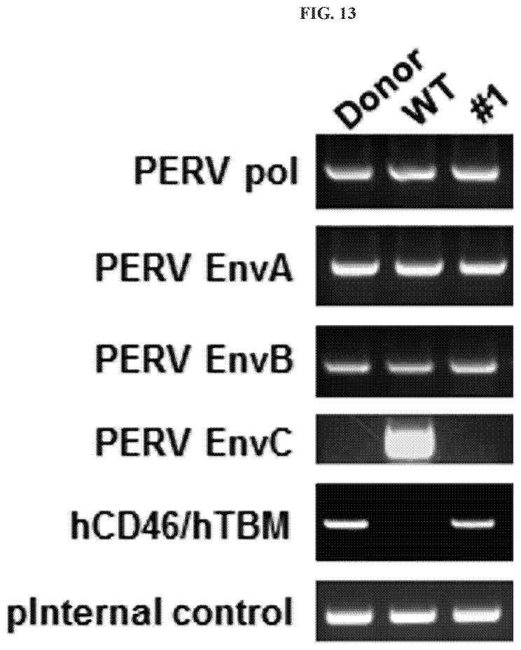

Further, in order to identify the nucleotide sequence of the transgenic pig, fibroblasts of the living individual were obtained. Then, the porcine endogenous retrovirus envelope C and absence or presence of transfection were analyzed. The results are shown in FIG. 13.

As shown in FIG. 13, it was identified that human CD46 and TBM genes were normally introduced into the fibroblast of the transformed porcine (#1) prepared in Example 5, and the porcine endogenous retrovirus envelope C was negative.

6-2. Validation of Transgenic Cloned Pigs

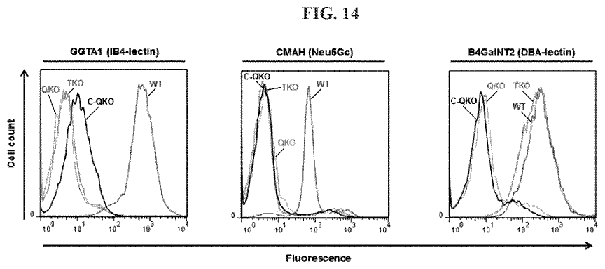

Blood-derived peripheral blood mononuclear cells (PBMCs) of transgenic cloned pig #1 identified in Example 6-1 were isolated, and then FACS analysis was performed on each lacked gene. More specifically, blood was collected from each of individuals of wild-type, TKO (GGTA1/CMAH/iGb3s triple knock-out), QKO (PERVc+GGTAl/CMAH/iGb3s/β4GalNT2 quadra knock-out) and C-QKO according to the present disclosure (PERV Envc-GGTA1/CMAH/iGb3s/β4GalNT2 quadra knock-out; hCD46/hTBM) using a syringe. The blood was diluted with DPBS at a 1:1 ratio. The diluted blood was put into ficoll-paque (GE healthcare) at 1:1 (volume/volume), and centrifugation was performed thereon at 500 g for 40 minutes. After separating the buffy coat layer in the middle, it was washed with DPBS and FACS analysis was performed using an antibody for each gene. The results are shown in FIG. 14.

As shown in FIG. 14, it was identified that the genes (GGTA1, CMAH, β4GalNT2) were normally deleted from PBMCs derived from the transgenic cloned pig (C-QKO) according to the present disclosure and thus no protein was produced, when compared to a control group.

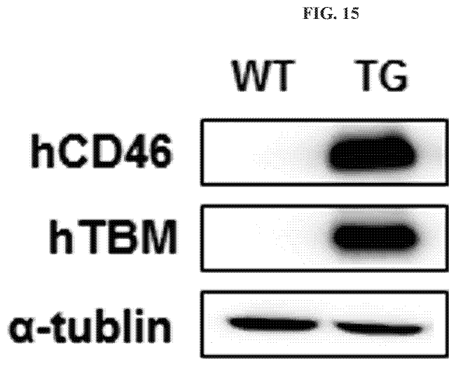

Further, after obtaining wild-type and transgenic cloned pig #1-derived ear fibroblasts, Western blot analysis thereon was performed to identify human CD46 and TBM protein expression. The results are shown in FIG. 15.

As shown in FIG. 15, it was identified that human CD46 and TBM genes were well generated in ear fibroblast (TG) derived from the transgenic cloned pig #1 according to the present disclosure.

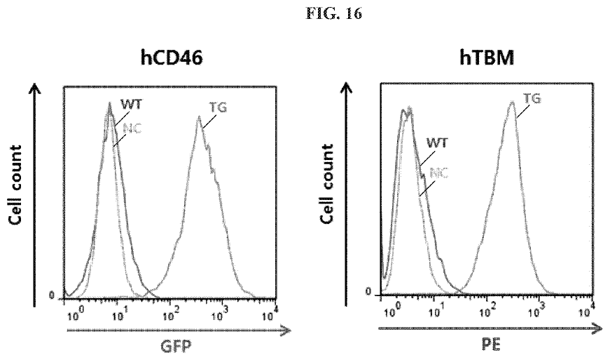

Additionally, to identify protein expression in each tissue, transgenic cloned pig #1-derived corneal endothelial cells were isolated, and FACS analysis thereon was performed using human CD46 and TBM antibodies. Specifically, wild-type and transgenic cloned pig-derived eyes were treated with 70% alcohol for 5 minutes to remove the integument, the limbus and cornea were removed, and then only the inner endothelial layer was cut to a size of 5 mm. After treatment thereof with 0.25% trypsin-EDTA solution for 30 minutes, the endothelial cells were separated therefrom by scraping the Emebraan van Descemet with a glass needle under microscope observation. After centrifugation thereof at 1500 rpm for 3 minutes, only the pellet was obtained and cultured. The cultured cells were subjected to cell immunostaining and FACS analysis using human CD46 and human TBM antibodies. The results are shown in FIG. 16.

As shown in FIG. 16, it was identified that fluorescence signals due to human CD46 and TBM genes were detected in the corneal endothelial cells (TG) derived from the transgenic cloned pig #1 according to the present disclosure, compared to the wild-type.

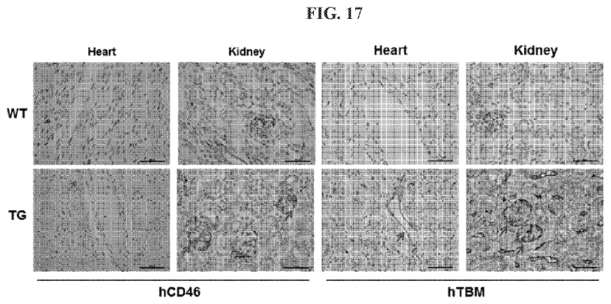

Next, after sacrificing individuals with the same genetic trait born from the same mother, tissue immunostaining was performed thereon. Specifically, the hearts and kidneys of the wild-type and the transgenic individuals were fabricated into paraffin blocks, and then deparaffinized. After blocking thereof, immunofluorescence staining thereon was performed using human CD46 and human TBM antibodies, and images were analyzed using a microscope and using DAB reagent. The results are shown in FIG. 17.

As shown in FIG. 17, it was identified that human CD46 and TBM proteins are DAB-positive in the transgenic cloned pig having the same genetic trait as that of the transgenic cloned pig #1 according to the present disclosure, compared to the wild type. In particular, strong positivity was identified in intracardiac blood vessels, myocardium, and renal glomeruli. Thus, based on this result, it was identified that human CD46 and TBM proteins were well expressed in muscle and blood vessels.

### Example 7. Functional Analysis of Transgenic Pigs in which GGTA1, CMAH, iGb3s and β4GalNT2 Genes are Removed and which Express Human CD46 and TBM Genes

7-1. Verification of APC (Activated Protein C)

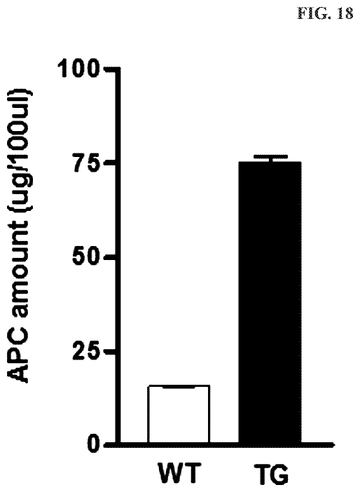

The human TBM gene combines with thrombin to create a thrombin-thrombomodulin composite and then activates protein C to act as an anticoagulant and anti-inflammatory agent. This could be a solution to the problem of blood coagulation that occurs during xenotransplantation. Accordingly, the amount of the protein C produced in transgenic cloned pigs was identified by quantifying the active protein C as known as a marker of anticoagulants. Specifically, after sacrificing a wild-type individual and an individual having the same genetic trait as that of the transgenic cloned pig (#1) according to the present disclosure, 10⁶ splenocytes were obtained therefrom. The obtained splenocytes were treated with human thrombin (Merck, Australia) and protein C (Merck, Australia) at 37° C. for 30 minutes, followed by treatment with hirudin (Merck, Australia) to stop the reaction. After rotating at 2000 rpm for 5 minutes to obtain a supernatant, the supernatant was dispensed into a 96-well plate. 1 mM spectrozyme PCa1 (American Diagnostica, USA) was applied thereto. A value was measured at a wavelength of 405 nm and at 37° C. using NanoQuant (Tecan) equipment. The results are shown in FIG. 18.

As shown in FIG. 18, it was identified that a larger amount of the active protein C was produced in the splenocytes of the transgenic pigs (TG) according to the present disclosure, compared to the wild type. Based on this result, it is expected that the survival period of the recipient will be increased as blood coagulation is inhibited by the production of the human TBM protein during xenotransplantation using transgenic pigs according to the present disclosure.

7-2. C3 Deposition Verification

The immune response due to complement activity after hyperacute immune rejection reaction during xenotransplantation should be controlled. It has been revealed that among the complement activity suppressor genes, hCD46 (Membrane Cofactor Protein; MCP) causes Factor I to bind to C3b or C4b of the complement activity component on the membrane, and Factor I and CD46 may inactivate the C3b to inhibit the complement activity.

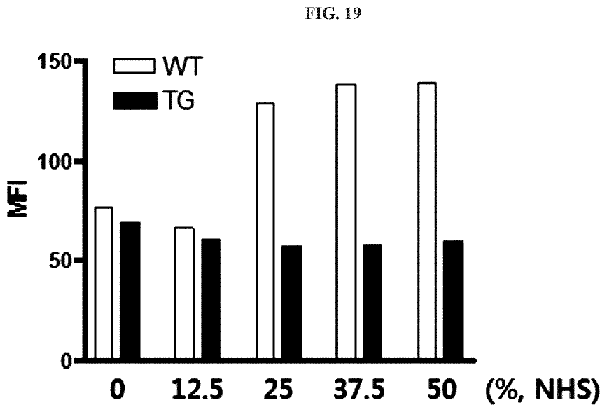

In this regard, after obtaining wild-type and transgenic cloned pig(#1)-derived ear fibroblasts, the obtained ear fibroblasts were treated with normal human serum (NHS) at varying concentrations such as 12.5%, 25%, 37.5%, and 50% for 2 days. Then, FACS analysis thereon was performed using the C3 antibody. The results are shown in FIG. 19.

As shown in FIG. 19, it was identified that a smaller amount of C3 deposition occurred in the ear fibroblast derived from the transgenic pig (TG) according to the present disclosure, compared to the wild type. Based on this result, it may be expected that in the transgenic pigs according to the present disclosure, C3 deposition is reduced due to human CD46 gene expression, such that complement activity is suppressed during xenotransplantation, and the survival period of recipients is expected to increase due to reduction of immune rejection reaction.

Comprehensively, based on the above experiment, it was identified that in the transgenic cloned pig according to the present disclosure, the porcine endogenous retrovirus EnvC was negative, and four genes, that is, GGTA1, CMAH, β4GalNT2 and iGb3s were knocked out using CRISPR-Cas9 as a gene scissors, and the transgenic cloned pig had the characteristics of expressing the human CD46 and TBM genes. Accordingly, the transgenic cloned pigs according to the present disclosure may not cause metastasis of porcine endogenous retrovirus that occurs in xenotransplantation, and at the same time, may overcome hyperacute and antigen-antibody-mediated immune rejection reaction, immune rejection reaction due to blood coagulation, immune rejection reaction due to complement activity. Thus, the transgenic cloned pigs according to the present disclosure may be usefully utilized as a donor animal for transplantation of heterogeneous organs and cells.

### Accession Number

Name of depository institution: Korea Cell Line Research Foundation

Accession number: KCLRF-BP-00464

βDeposit date: 2019 Jan. 30

1. A transformed cell for preparing a transgenic cloned pig for xenotransplantation,
wherein the transformed cell has a recombinant vector for knocking out GGTA1 (Alpha 1,3-Galactosyltransferase), a recombinant vector for knockout of CMAH (CMP-N-acetylneuraminic acid hydroxylase), a recombinant vector for knocking out iGb3s (Isoglobotrihexosylceramide synthase), a recombinant vector for knocking out β4GalNT2 (Beta-1,4-N-Acetyl-Galactosaminyl Transferase2), a recombinant vector for expressing human CD46, and a recombinant vector for expressing human TBM (Thrombomodulin) introduced thereto,
wherein in the transformed cell, PERV (Porcine Endogenous Retrovirus) EnvC (Envelope C) is negative.

2. The transformed cell of claim 1, wherein the recombinant vector for knocking out the GGTA1 recognizes exon #4 of porcine chromosome 1 and knocks out the GGTA1 gene.

3. The transformed cell of claim 1, wherein the recombinant vector for knocking out the CMAH recognizes exon #9 of porcine chromosome 7 and knocks out the CMAH gene.

4. The transformed cell of claim 1, wherein the recombinant vector for knocking out the iGb3s recognizes exon #4 of porcine chromosome 6 and knocks out the iGb3s gene.

5. The transformed cell of claim 1, wherein the recombinant vector for knocking out the β4GalNT2 recognizes exon #1 of porcine chromosome 12 and knocks out the β4GalNT2 gene.

6. The transformed cell of claim 1, wherein the recombinant vector for expressing the human CD46 has a vector map shown in FIG. 2.

7. The transformed cell of claim 1, wherein the recombinant vector for expressing the human TBM (Thrombomodulin) has a vector map shown in FIG. 3.

8. The transformed cell of claim 1, wherein the transformed cell has an accession number KCLRF-BP-00464.

9. A method for preparing a transgenic cloned pig for xenotransplantation, the method comprising:
a step of transplanting the transformed cell according to claim 1 into an enucleated oocyte to prepare a nuclear transferred oocyte; and
a step of transplanting the nuclear transferred oocyte into a fallopian tube of a surrogate mother.

10. A transgenic cloned pig for xenotransplantation.
